(19) United States
(12) Patent Application Publication
(10) Pub. No.: US 2018/0036162 A1
(43) Pub. Date: Feb. 8, 2018

(54) SHOCK ABSORBER INSERT FOR AN ORTHOPEDIC WALKING BOOT

(71) Applicant: ORTHO SYSTEMS d/b/a OVATION MEDICAL, Agoura Hills, CA (US)

(72) Inventors: Steven L. HECKER, Los Angeles, CA (US); Tracy E. GRIM, Thousand Oaks, CA (US); Kenji WATABE, Ventura, CA (US); Ryan C. COHN, Torrance, CA (US); Tim CROWLEY, Ventura, CA (US); Veneza YUZON, Calabasas, CA (US)

(21) Appl. No.: 15/706,324

(22) Filed: Sep. 15, 2017

Related U.S. Application Data
(62) Division of application No. 14/214,205, filed on Mar. 14, 2014.
(60) Provisional application No. 61/802,024, filed on Mar. 15, 2013.
(60) Provisional application No. 61/916,086, filed on Dec. 13, 2013.

(51) Int. Cl. A61F 5/058

(52) U.S. Cl. CPC A61F 5/0585

(57) ABSTRACT

An orthopedic walking boot, including a base to support a user's foot; a support assembly extending from the base to support the user's lower leg; an outer sole; and a shock absorber insert arranged with the base and the outer sole to provide shock absorption to the user's foot.

This present application is a divisional of, and claims priority to, U.S. Non-Provisional Application Ser. No. 14/214,205, filed Mar. 14, 2014, entitled “SHOCK ABSORBER INSERT FOR AN ORTHOPEDIC WALKING BOOT,” now pending, which claims the benefit of and priority to U.S. Provisional Patent Application Ser. Nos. 61/802,024, filed Mar. 15, 2013 and 61/916,086, filed Dec. 13, 2013, each of which are incorporated herein by reference in their entirety as if fully set forth herein.

### FIELD

The present disclosure relates generally to orthopedic walking boots.

### BACKGROUND

It is common that people, especially active and/or frail people, experience a variety of lower leg and ankle injuries. To aid in the treatment of the injuries it is desirable to immobilize the injury, typically above and below the affected joint.

Physicians traditionally place a patient's leg in a short leg cast, which is a cast that begins at the patient's toes and ends below the patient's knee. Generally, casts retain heat, cause an itching sensation on the skin, and rub against the leg after swelling of the leg subsides.

An alternative to the short leg cast is an orthopedic walking boot, or a premanufactured orthopedic walking boot, that is made of a rigid plastic frame lined with a soft component (e.g., a soft padding) to accommodate the leg comfortably. Often, the liner, or soft component, may house a series of air bladders that can be adjusted by the patient to improve the fit and help compress the swelling to reduce pain and increase stability. The orthopedic walking boots can be removed to treat skin problems, such as, to remove sutures or conduct passive range of motion exercises. Short leg casts do not offer the luxury of easy on/off.

An orthopedic walking boot is primarily a rigid encasing that envelopes the leg and immobilizes the foot and ankle at a neutral position (e.g., the foot extends 90 degrees relative to the leg). The patient can walk easiest if the ankle is fixed at 90 degrees. At angles other than 90 degrees the patient will be walking on the toes or on the heel. The sole of the foot is generally curved from front to back in a rocker bottom fashion. The curvature of the sole provides a smoother stride from front to back allowing the heel to strike the ground first, followed by a rocking of foot forward, and finally a push off on the toes for a successful step.

### SUMMARY

Aspects of an orthopedic walking boot may include a base to support a user's foot; a support assembly extending from the base to support the user's lower leg; an outer sole; and a shock absorber insert arranged with the base and the outer sole to provide shock absorption to the user's foot.

The shock absorber insert may be arranged along a section of the base configured to support the user's plantar portion of the heel. The shock absorber insert may be located between the base and the outer sole. The shock absorber insert may include a plurality of ribs and the outer sole comprises a plurality of apertures. The ribs may extend into the apertures. The outer sole may include an outer surface. The ribs may extend through the apertures beyond the outer surface. The shock absorber insert may be located in the pocket. The shock absorber insert may include a perimeter rib. The pocket may comprise a channel configured to mate with the perimeter rib. The shock absorber insert may include a plurality of apertures. The outer sole may extend through the apertures towards the base. The outer sole may include a durometer, and the shock absorber insert comprises a durometer lower than the outer sole's durometer. The shock absorber insert may include a plurality of sections. Each of the sections may comprise a different durometer. The shock absorber insert may include a plurality of discrete shock absorbing elements. The base may include a plurality of apertures cooperating with the shock absorber insert. The base may include a pocket and the absorber insert may be located in the pocket. The shock absorber insert may include a perimeter rib, and the pocket may include a channel configured to mate with the perimeter rib. The shock absorber insert may include a plurality of apertures, and the outer sole may extend through the apertures towards the base. The outer sole may include a durometer, and the shock absorber insert may include a durometer lower than the outer sole's durometer. The shock absorber insert may include a plurality of sections, where each of the sections comprises a different durometer. The shock absorber insert may include a plurality of discrete shock absorbing elements.

Another aspect of an orthopedic walking boot may include a base to support a user's foot; a support assembly extending from the base to support the user's lower leg; an insole plate; and one or more shock absorber pins between the base and the insole plate to provide shock absorption to the user's foot.

The base may include a footbed having one or more apertures, each of the one or more shock absorber pins may be supported by a different one of the apertures. Each of the one or more shock absorber pins may include a head and a shaft. The shaft for each of the one or more shock absorber pins may be inserted into the aperture supporting it with the head engaging the insole plate. Each of the one or more shock absorber pins may include a head having a spherical, a cylindrical donut, a pyramid, a trapezoidal, or a serrated trapezoidal shape.

### BRIEF DESCRIPTION OF THE DRAWINGS

FIG. 1 is perspective view of an orthopedic walking boot according to aspects of the present invention;

FIG. 2 is an exploded perspective view of the base, outer sole, and shock absorber insert of the orthopedic walking boot of FIG. 1;

FIG. 3 is a partially exploded perspective view of the base, outer sole, and shock absorber insert of FIG. 1;

FIG. 4 is a cut perspective view of the base, outer sole, and shock absorber insert of FIG. 1;

FIG. 5 is an exploded view of the base and the shock absorber insert of FIG. 1;

FIG. 6 is an exploded perspective view of the base, insole plate, and shock absorber pins, of an orthopedic walking boot, in accordance with other aspects of the present invention;

FIG. 7 is a front cut view of the base of FIG. 6;

FIG. 8 is a partially exploded perspective view of an insole plate in accordance with aspects of the present invention;

FIG. 9A-9E are perspective view of shock absorber pins in accordance with aspects of the present invention.

### DETAILED DESCRIPTION

Various aspects of the present invention will be described herein with reference to drawings that are schematic illustrations of idealized configurations of the present invention. As such, variations from the shapes of the illustrations as a result, for example, manufacturing techniques and/or tolerances, are to be expected. Thus, the various aspects of the present invention presented throughout this disclosure should not be construed as limited to the particular shapes of elements (e.g., regions, layers, sections, substrates, etc.) illustrated and described herein but are to include deviations in shapes that result, for example, from manufacturing. Thus, the elements illustrated in the drawings are schematic in nature and their shapes are not intended to illustrate the precise shape of an element and are not intended to limit the scope of the present invention, unless intentionally described as such.

It will be understood that when an element such as a region, layer, section, or the like, is referred to as being “on” another element, it can be directly on the other element or intervening elements may also be present. In contrast, when an element is referred to as being “directly on” another element, there are no intervening elements present. It will be further understood that when an element such as a structure is referred to as being coupled to another element, it can be directly connected to the other element or intervening elements may also be present. Similarly, two elements may be mechanically coupled by being either directly physically connected, or intervening connecting elements may be present. It will be further understood that when an element is referred to as being “formed” on another element, it can be deposited, attached, connected, coupled, or otherwise prepared or fabricated on the other element or an intervening element.

Furthermore, relative terms, such as “lower” or “bottom” and “upper” or “top,” may be used herein to describe one element's relationship to another element as illustrated in the drawings. It will be understood that relative terms are intended to encompass different orientations of an apparatus in addition to the orientation depicted in the drawings. By way of example, if the orientation of an orthopedic walking boot shown in the drawings is turned over, elements described as being on the “lower” side of other elements would then be oriented on the “upper” side of the other elements. The term “lower”, can therefore, encompass both an orientation of “lower” and “upper,” depending of the particular orientation of the orthopedic walking boot. Similarly, if the orientation of an orthopedic walking boot shown in the drawing is turned over, elements described as “below” or “beneath” other elements would then be oriented “above” the other elements. The terms “below” or “beneath” can, therefore, encompass both an orientation of above and below.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and this disclosure.

It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. The term “and/or” includes any and all combinations of one or more of the associated listed items.

The detailed description set forth below in connection with the appended drawings is intended as a description of various aspects of the present invention and is not intended to represent all aspects in which the present invention may be practiced. The detailed description includes specific details for the purpose of providing a thorough understanding of the present invention. However, it will be apparent to those skilled in the art that the present invention may be practiced without these specific details. In some instances, well-known structures and components are shown in block diagram form in order to avoid obscuring the concepts of the present invention.

Various aspects of the present invention may provide an orthopedic walking boot that may be fitted around the leg to provide support and allow ambulation for the affected limb.

Reference to various ranges may be used to describe certain aspects of the present invention. By way of example, a range may be used to describe variations of the bonding force at different points on an outer sole to describe an evenly distributed bonding of the outer sole to the base of the orthopedic walking boot. By way of an example, an outer sole which provides evenly distributed bonding to the base of the orthopedic walking boot may exhibit a narrower tolerance band of force values at all x,y coordinates on the bonding surface than tolerance band of any other attachment method of the outer sole to the base of the orthopedic walking boot.

People often experience injuries to the lower leg and ankle. For example, blunt trauma, sports injuries and common falls are the primary causes. Injuries such as fractures of the bones or soft tissue injuries (e.g., ligamentous tears) have similar symptoms. Swelling, pain and inability to ambulate without support are expected and predictable. Some injuries need to be immobilized for a period of time for the injury to heal. The time required for ligamentous injuries to heal is similar to the time required for fractures to heal. A period of 4 to 6 weeks of immobilization is common. Different injuries require different rehab times and regimes.

Aspects of the present invention are directed to orthopedic walking boots. In an aspect of the prevention invention, an orthopedic walking boot may include bilateral struts which connect a base of the orthopedic walking boot to an upper portion of the orthopedic walking boot. The struts may be rigid and provided on either side of the leg. The bilateral struts may be held onto the limb with strapping systems that encircle at least a portion of the limb. In another aspect, the base may be attached a posterior piece which extends from the foot to the back of the leg and calf forming a clamshell configuration. In the clamshell configuration, a single piece encompasses the side of the leg (similar to the bilateral configuration) as well as the rear of the leg. The orthopedic walking boot may include an adjoining anterior piece that joins or overlaps the posterior piece and is held on by a traditional strapping system or with mechanical attachment mechanism. In another aspect, the orthopedic walking boot may comprise a “hybrid” configuration (also referred herein as a “multi-sectioned” configuration). In the hybrid configuration, the base may be attached to the bilateral struts of the bilateral configuration and also attached a separate/non-integral posterior element that encompasses the rear of leg (similar to the rear portion of the clamshell). In this manner, the bilateral struts surround the side of the legs while the separate posterior portion encompasses the rear of the leg. Thus, the hybrid configuration achieves a similar result as the clamshell with multiple sections, hence, “multi-sectioned.”

In an aspect, the orthopedic walking boot may be configured such that the portion that receives the user's foot (e.g., the base portion) extends at 90° degrees or at substantially 90° relative to a longitudinal axis of the portion that receives the user's leg (e.g., the upper portion). In another aspect, the orthopedic walking boot may include two struts rising from the base. The orthopedic walking boot may further include a soft component within the constraints of the struts and on top of the base. The soft component may be held by straps.

Patients who have recently been placed into an orthopedic walking boot by their doctor likely experienced acute trauma in their lower extremities, and may experience swelling and in many cases, intense pain. For these new patients, each step in an orthopedic walker may bring strong discomfort. With each stride of the patient, the orthopedic walking boot contacts the floor with a particular speed and momentum, creating a certain amount of deceleration force. The higher the downward velocity of the walker boot just before impact, the larger the impulse reaction, defined as the change of acceleration over time. Injured patients impact acutely, as it almost always results in pain to the injured area.

In an orthopedic walking boot there are generally two discrete zones that transfer the force of an impact to the wearer. One zone relates to the impact between the outer sole of the walker and the floor surface. The second zone relates to the impact between the foot of the patient and the insole of the walker boot. Both zones are important when considering patient pain.

An effective shock absorbing system may function without any degradation of primary functional aspects of the walker such as gait and overall mobility. In addition, key mechanical properties of the outer sole such as wear resistance and grip may be maintained in the regions of the walker that require these properties.

The amount of impact that can be absorbed by the base and outer sole may depend on several factors, some of which include the patient's weight, the durometer of each material included in the assembly, position of the shock element on the walker base as well as other factors.

As will be described in further detail below, the primary impact zone is located at the heel section of the outer sole, and absorbs the impact of the walker boot as the heel section of the outer sole strikes the surface upon which the patient is walking.

One aspect of the present invention creates a hybrid strike zone by combining two parts together into one assembly comprising the soft shock absorbing qualities of a lower durometer material with the wear resistant qualities of a higher durometer material. The materials may be in any number of material categories, including but not limited to thermoplastic elastomer (hereinafter “TPE”), thermoplastic polyurethane (hereinafter “TPU”), thermoplastic vulcanizate (hereinafter “TPV”), silicones, rubbers, gels, and the like.

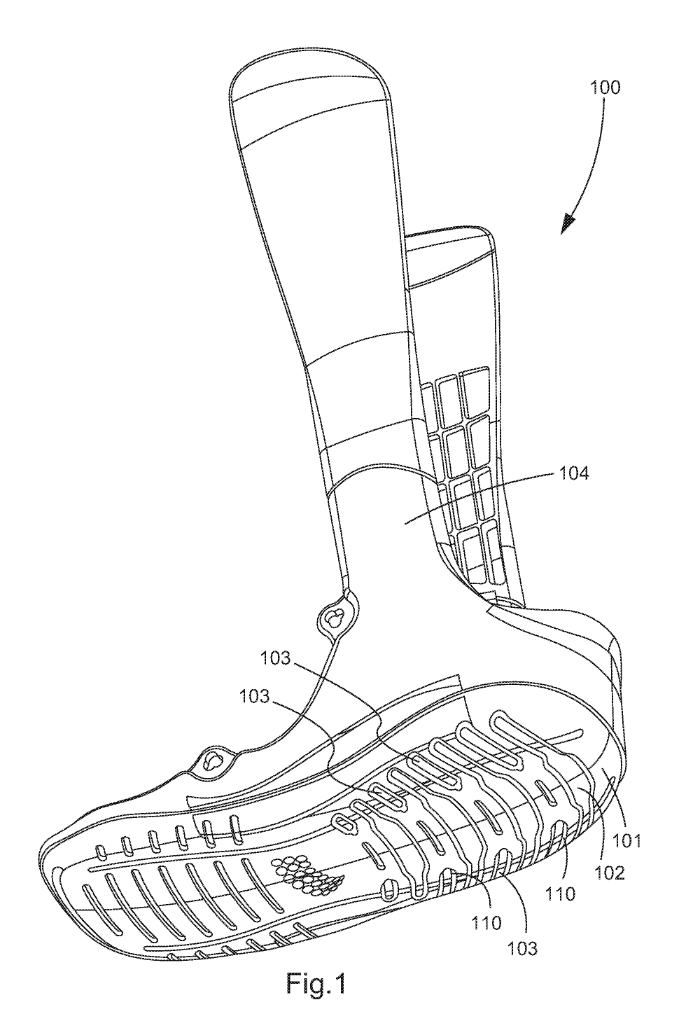

FIG. 1 is a perspective view of an orthopedic walking boot 100. The orthopedic walking boot 100 may include a high durometer elastomer outer sole 101 partially covering a softer durometer shock absorber insert 102 (viewable through apertures 110 in the outer sole), and a base 104. In one aspect, the shock absorber insert 102 may be molded separately from the outer sole and then may be attached to the base 104 by a conventional method such as with adhesive. In another aspect a plurality of ribs 103 may extend from the shock absorber insert 102. As shown in FIG. 1, the ribs 103 may be exposed through the outer sole 101 via apertures 110 formed in the outer sole, and may be positioned above, below or at the elevation of the outer sole 101. For example, in the configuration where the ribs 103 extend through the apertures 110 of the outer sole 101, the shock absorber insert 102 and ribs 103 would together provide additional grip in wet or slippery conditions.

In another aspect, the shock absorber insert 102 may be located outside of the outer sole 101. In a further aspect, the shock absorber insert 102 may be located on a front and/or rear section of underside of the base 104. In yet another aspect, the orthopedic walking boot may include a plurality of separate/distinct shock absorber inserts attached to the outer sole. Furthermore, the shock absorber insert 102 may comprise a plurality of sections having various durometers. In another aspect, the shock absorber insert may be replaceable to allow for the use of different shock absorber inserts having different durometers. The ability to replace the shock absorber insert having different durometers accommodates different weights of patients with the same base. The shock absorber insert 102 comprise a substantially lower durometer than the outer sole 101 and may be permanently attached to the base 104 by various methods including, but not limited to, overmolding, adhesives, interference fits, mechanical fasteners, welding, and the like. In an example aspect the outer sole may have a Shore A durometer of 60 A and the shock absorber insert may have a Shore A durometer of 30.

In one aspect, the shock absorber insert may be disposed completely inside the outer sole and is not visible to the consumer (e.g., if the outer sole does not have apertures). Additionally, the a shock absorber insert may be located in the general area of the heel, in the general area of the toes or front of the foot, or may be configured as a plurality of shock absorber inserts, located anywhere on the walking surface of the orthopedic walking boot. Furthermore, a distribution of a plurality of discrete shock absorber inserts may be envisioned, where a shock absorber insert in one area of the walker is complemented by other shock absorber inserts of different durometers, to address for instance, the different shock absorbing requirements in different zones of the walker.

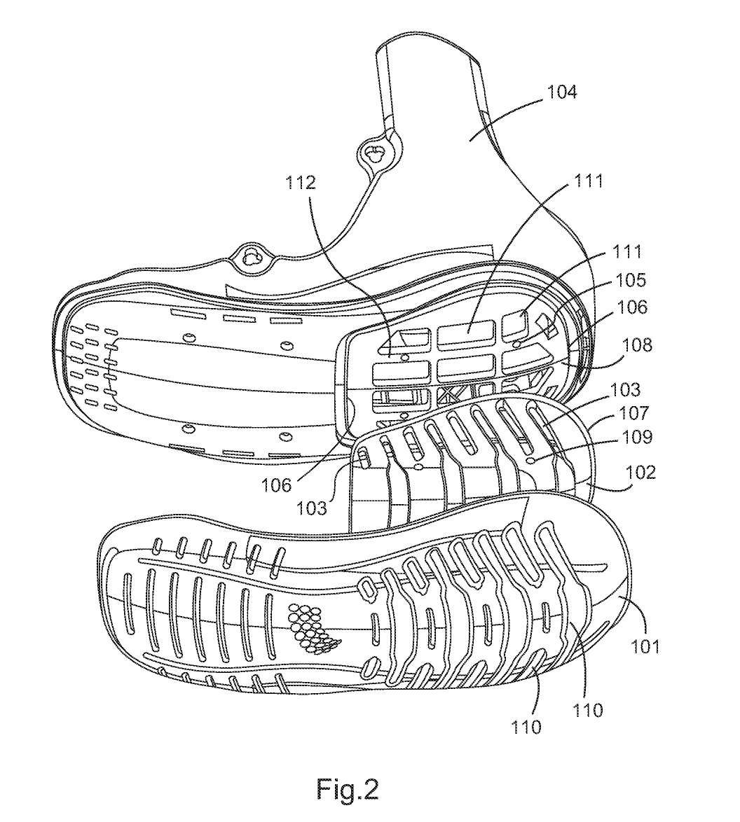

FIG. 2 is an exploded perspective view of the base 104, the shock absorber insert 102, and the outer sole 101. As noted above, the shock absorber insert 102 may have a lower durometer than the outer sole 101. As shown in FIG. 2, the base 104 may include a pocket 108 for receiving the shock absorber insert 102. The base may further include through holes 111. As shown in FIG. 2, the through holes 111 may be provided throughout the area of the pocket 108. Example through holes 111 may have a rectangular, square shape, among other geometries. As shown in FIG. 2, the through holes 111 may be disposed such that crossbars 112 are defined between the through holes 111. The perimeter of the pocket 108 may be defined by a rib and a channel 106. As shown in FIG. 2, channel 106 may define a shape corresponding to the shape of the shock absorber insert 102. For example, the shock absorber insert 102 may have a rib or lip 107 that may align with the channel 106 of the base 104. The shock absorber insert 102 may be placed within pocket 108 by inserting the lip 107 of the shock absorber insert 102 within the channel 106.

The shock absorber insert 102 may include one or more through holes 109, while the base 104 may include a corresponding number of through holes 105. The through holes 105, 109 may be disposed such that when the shock absorber insert 102 is placed in the pocket 108, the holes 105, 109 align. With this configuration, when the outer sole 101 is overmolded onto the base 104, the molten resin of the outer sole will flow through the holes 105, 109, and harden into a shape resembling the geometry of a nail, thereby securing the shock absorber insert 102 between the outer sole 101 and the base 104. The overmolding process may also prevent the shock absorber insert 102 from moving out of position due to the chemical bond between the outer sole 101 and the base 104. An additional utility of the perimeter rib/groove system is that it creates a sealing surface which prevents the molten overmold material used to create the outer sole from passing around the shock absorber insert and into the cavity of the base.

In another aspect, the shock absorber insert may not have through holes, in which case corresponding through holes need not be present on the base. In this aspect, the shock absorber insert may be coupled to the base within the pocket by an adhesive.

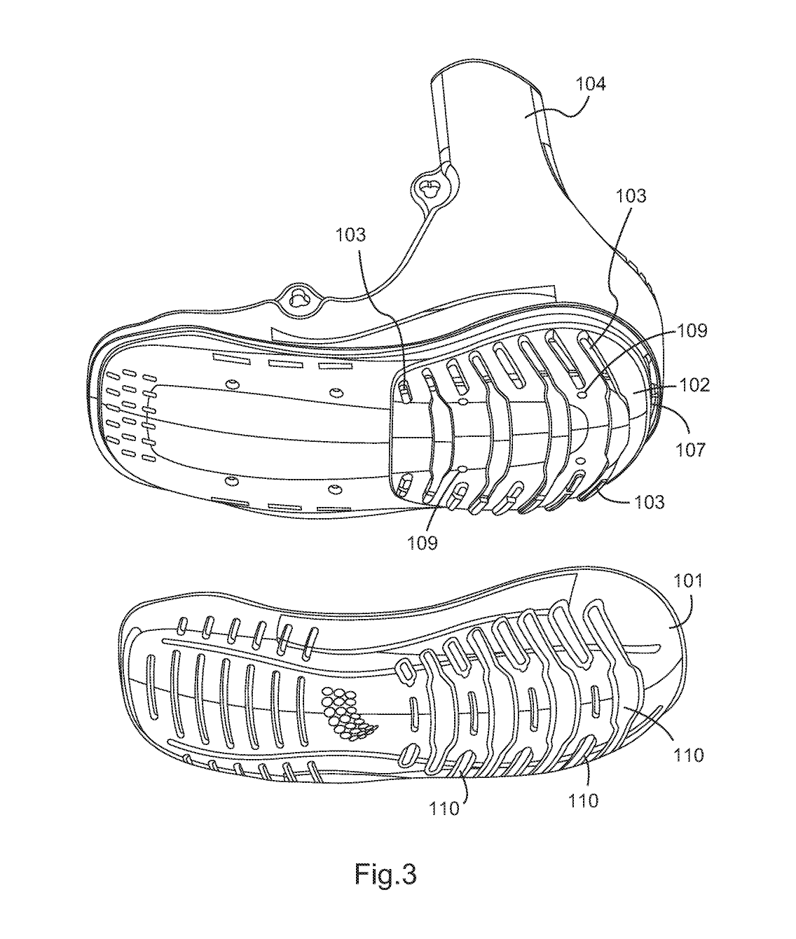

In another aspect, the portion of the base surrounding the through holes 111, for example the crossbars 112, may include projecting posts (not shown). The projecting posts may extend from the underside surface of the base (i.e., the surface shown in FIG. 2) in a direction away from the user's foot (i.e., toward the outer sole in FIG. 2). Similarly, the upper side surface of shock absorber insert (i.e., the surface facing the base when the shock absorber insert is disposed in the pocket) may include corresponding receiving elements that are mateable with the posts. Thus, in such an aspect, when the shock absorber insert is disposed within the pocket (as shown in FIG. 3), the receiving elements of the shock absorber insert may align with the posts, and the posts may extend into the receiving elements. This arrangement may provide additional stability and ensure that the shock absorber insert remains in position. Furthermore, adhesive may be applied to the tips of the posts prior to mating with the receiving elements to provide further stability.

FIG. 3 shows a partial exploded view of the base 104 having the shock absorber insert 102 inserted into the pocket. The outer sole 101 is shown exploded from the base 104. The shock absorber insert 102 may have a substantially lower durometer than the outer sole 101 and may be permanently attached to the base 104 by overmolding, adhering, using an interference fits, mechanical fasteners, welding, and the like. As noted above, the base may include posts and the shock absorber insert may include receiving elements to receive the posts. The outer sole 101 may include features intended to engage with similar features on the shock absorber insert 102. The outer sole 101 may be attached to the rigid base 104 and the shock absorber insert 102.

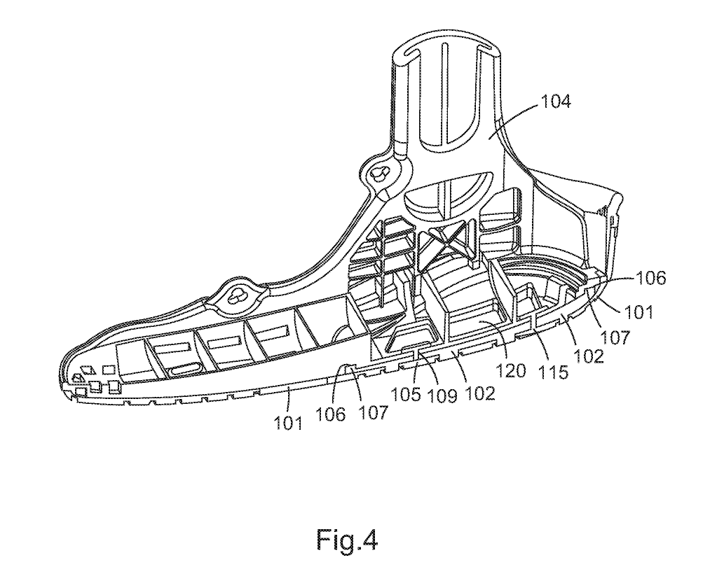

FIG. 4 shows a perspective cut view of the base 104 with a shock absorber insert 102 mated thereon. As shown in FIG. 4, the base 104 includes a channel 106 for receiving the lip 107 of the shock absorber insert 102. Specifically, FIG. 4 shows the shock absorber insert 102 placed against a bottom surface of the base 104 with the lip 107 of the shock absorber insert 102 inserted into the channel 106. With the shock absorber insert inserted into the channel of the base, the combined structure may be placed into an overmold injection tool, where after the liquid resin material of the outer sole may be injected into the mold. As the resin hardens it may form a chemical bond with the bottom surface of the base, as well as encapsulating the shock absorber insert between the base and the outer sole. Additionally, as noted above, the aligned through holes 105, 109 of the shock absorber insert and walker base may allow the molten material from the overmold to pass through the holes 105, 109, to form a mechanical interlock bond 115. The combination of outer sole, shock absorber pad, and base, is best seen in FIG. 4.

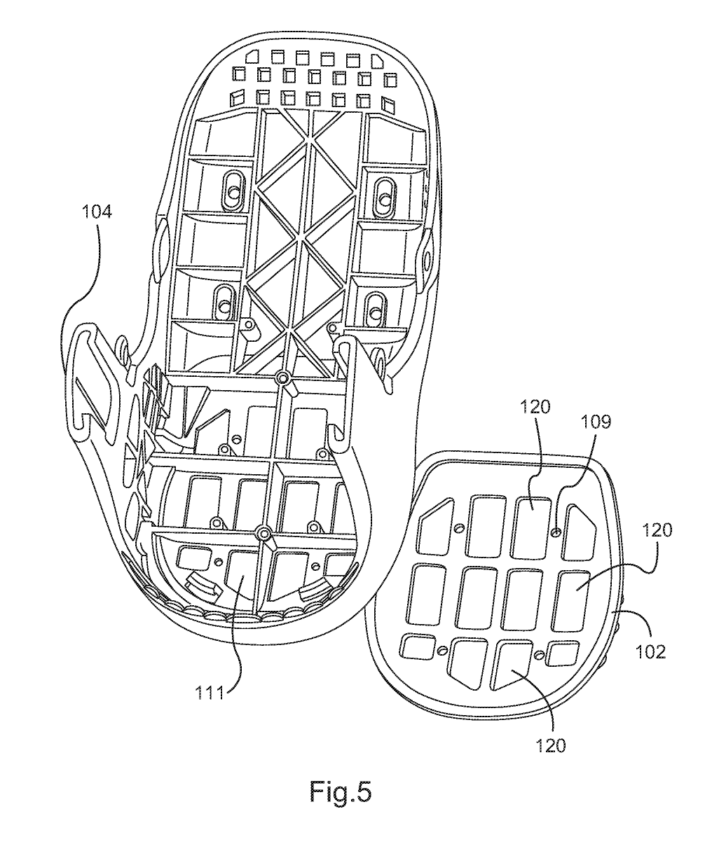

In another aspect, as best seen in FIG. 5, the shock absorber insert 102 may include local cavities 120 which may be cored out from the foot-side of the part. FIG. 5 shows a top exploded view of the base 104 and the shock absorber insert 102, with the outer sole omitted. As can be seen in FIG. 5, the cavities 120 may be shaped and disposed to correspond to the shape and position of the apertures 111 of the base 104. Once the shock absorber insert 102 is inserted into the underside pocket of the base 104, the cavities 120 of the shock absorber insert 102 may align with the apertures 111 of the base. The combination of aperture and cavity creates a space between the bottom of the base and the top of the shock absorber insert. This arrangement allows more deflection in the final product when worn, and allowing the softer material to remain in place during the high pressures typically encountered during the overmolding process. When an overmolding process is used to add the outer sole to the base, steel fingers located on the core side of the overmold tool that mate with the cavities 120 in the shock absorber insert 102 may serve to support the softer structure of the shock absorber insert 102 from collapsing during overmolding.

Another impact zone that affects patient pain is referred to as the secondary impact zone. Previously disclosed configurations have included an insole plate which is attached to the inside foot-bed of a rigid walker base. The insole plate has features a thickness of closed cell foam adhesively attached to the patient-facing surface, which cushions the foot of the injured patient. While this type of approach can absorb some of the impact imparted to the leg of the patient from contact with the insole plate, these foam cushions can experience a “compression set” which is a permanent deformation of the foam over time. The deformation over time reduces the efficacy of the secondary shock absorbing system. Furthermore, similar to automobile suspensions, a good shock absorbing system may include multiple shock absorbing areas.

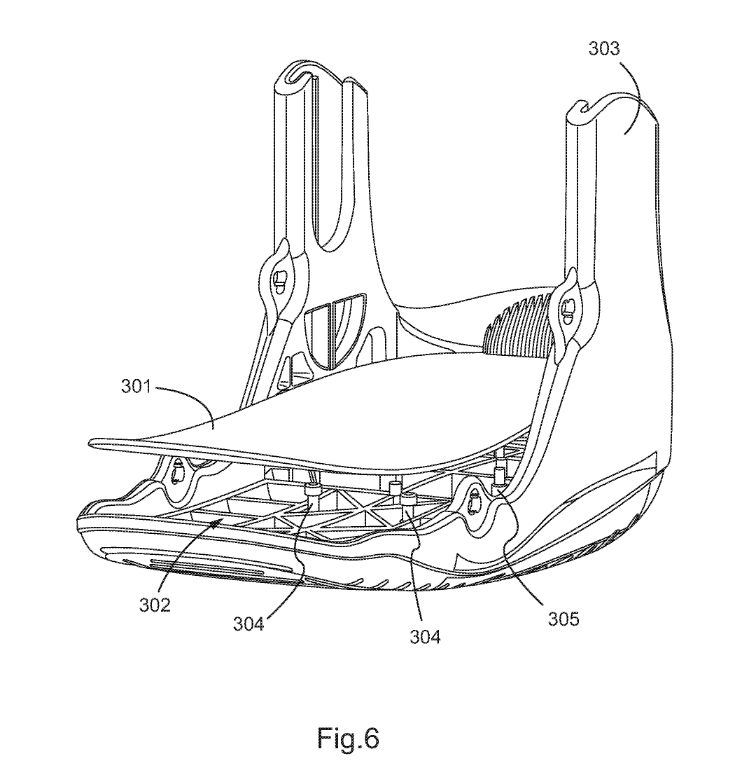

FIG. 6 shows an exploded perspective view of a portion of an orthopedic walking boot in accordance with another aspect of the present invention. The orthopedic walking boot may include a base 303, a rigid plastic insole plate 301 and an inside footbed 302. The insole plate 301 may be attached to the inside footbed 302. A plurality of shock absorber pins 304 comprising soft material, may be placed in a plurality of receivers 305 in a space between the bottom surface of the base 303 and the insole plate 301. As the boot strikes the walking surface of a floor during use, the wearer's foot applies pressure to the insole plate 301, which applies a force to the one or more shock absorber pins 304. The performance of the shock absorbing system is related to the durometer of the material of the shock absorbing element, the size of the element, the number of pins, the density and distribution of the pins and the geometry of the contact head of the pins, among other factors.

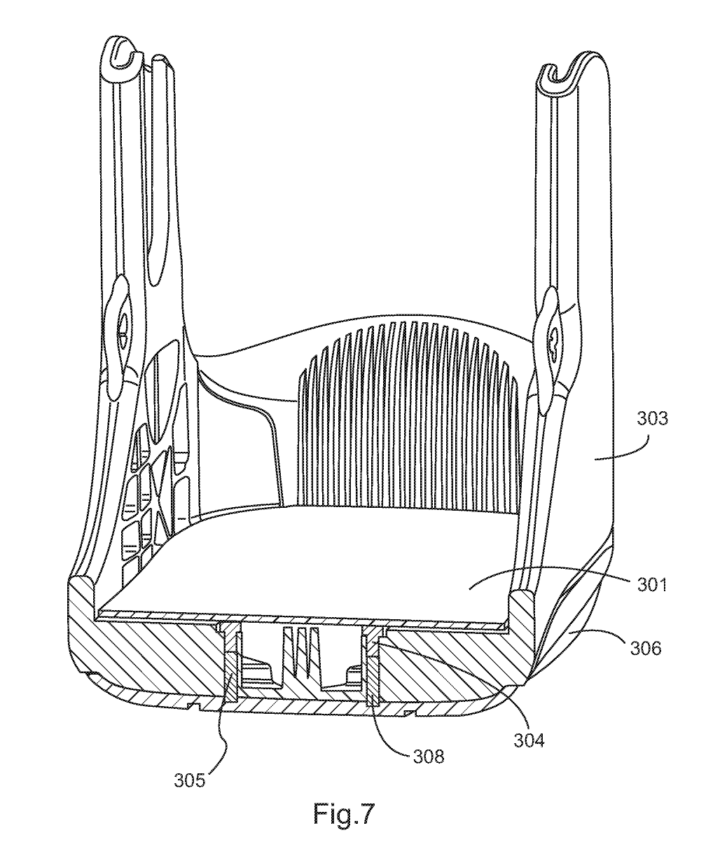

In one aspect, the shock absorber pins 304 may be manually inserted into a corresponding receiver 305 located proximally to the footbed 302. The pins may be permanently attached via by adhesive bonding, mechanical fastening, interference fits, and the like. In another aspect, the pins may be formed as part of the process of overmolding the outer sole to the base. FIG. 7 shows such a front cut view of the base 303 and an insole plate 301 supported by shock absorber pins 304 that have formed as part of the overmolding process. When overmolding the outer sole 306 to the base 303, the liquid resin may flow through a hole 308 in the base (similar to the holes 105 described above) and up into the receiver 305. The resin may continue to flow through the receiver 305 and out the open top of the receiver 305 to form the absorber pin 304.

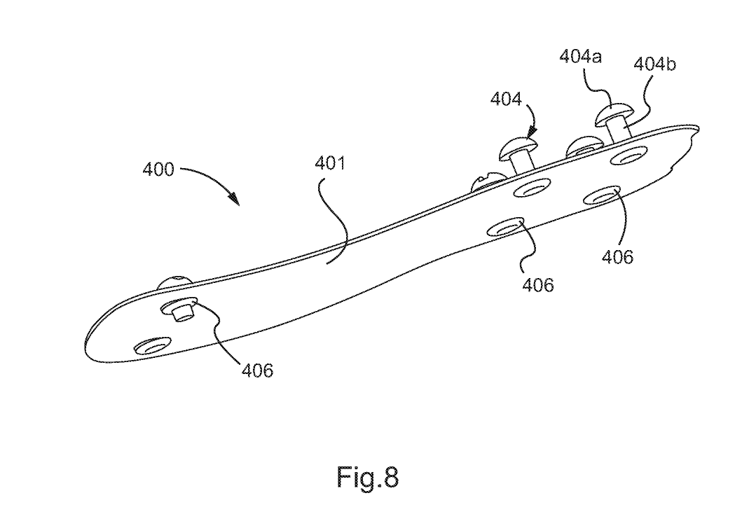

FIG. 8 shows a partially exploded perspective view of an insole plate 401 in accordance with another aspect of the present invention. As shown in FIG. 8, securing elements 404 having a head 404a and a shaft 404b may pass through the insole plate 401. The securing elements 404 may be arranged that the head 404a contacts an upper surface of the insole plate (i.e., the surface facing the foot). The shaft 404b may extend through the insole plate and be arranged such that the shaft 404b is received by a receiver (such as the receivers 305 shown in FIG. 6). As shown in FIG. 8, shock absorber rings 406 having a donut shape may be secured to an underside surface of the insole plate (i.e., the surface of the insole plate facing the ground). In an aspect, the shock absorber rings 406 may be overmolded in a separate operation onto the underside surface of the insole plate 401, around the hole that the shaft 404b passes through. In this configuration, the donut-shock absorber rings 406 may serve as gaskets to cushion the shock, and helps to preload the fasteners 406 that attach the insole plate to the walker base. A cushioning pad may be optionally assembled to the insole plate 401, and the resulting insole plate 401 assembly may be permanently fastened to the walker base footbed.

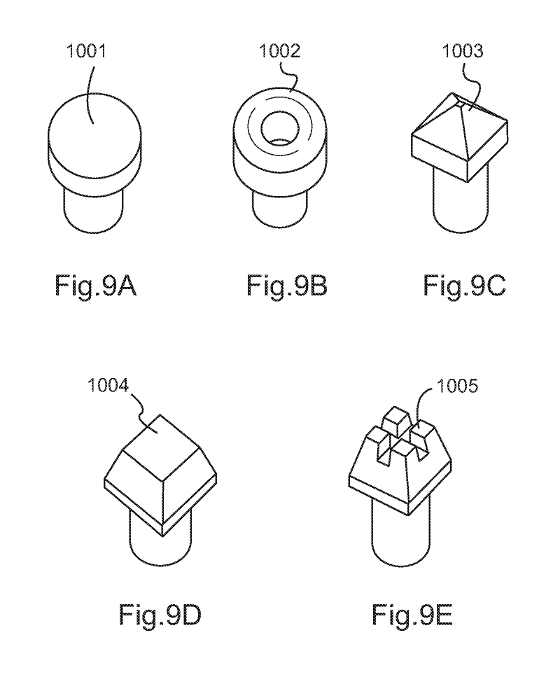

FIGS. 9A-9E show perspective of various aspects of the shock absorber pins. In particular, each of the FIGS. 9A-9E shows an absorber pin having different shaped contact heads. Although specific geometry of the contact head may be greatly varied, several of the preferred examples are illustrated in FIGS. 8A-8E. Various head geometries create various deceleration profiles, where a wider head produces a steeper deceleration slope, and a thinner or more pointed head produces a more gradual deceleration. Example geometries featured for the head of the shock absorber pin include a generally spherical head 1001, a cylindrical donut-type head 1002, a pyramid shaped head 1003, a trapezoidal head 1004 and a serrated trapezoidal head 1005. Other head geometries that may vary depending on the desired result, as well as multiple combinations of head geometry types in a single insole plate.

The claims are not intended to be limited to the various aspects of this disclosure, but are to be accorded the full scope consistent with the language of the claims. It is noted that specific illustrative embodiments of the invention have been shown in the drawings and described in detail hereinabove. It is to be understood that various changes and modifications may be made without departing from the spirit and scope of the invention. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.”

What is claimed is:

1. An orthopedic walking boot, comprising:
a base to support a user's foot;
a support assembly extending from the base along the user's lower leg and configured to support the user's lower leg;
a rigid insole plate extending substantially across a length of the base;
an outer sole; and
a shock absorber insert arranged between the insole plate and the outer sole to provide shock absorption to the user's foot.

2. The orthopedic walking boot of claim 1, wherein the shock absorber insert is arranged along a section of the base configured to support the user's plantar portion of the heel.

3. The orthopedic walking boot of claim 1, wherein the shock absorber insert comprises a plurality of ribs and the outer sole comprises a plurality of apertures, and wherein the ribs extend into the apertures.

4. The orthopedic walking boot of claim 1, wherein the base comprises a pocket, and wherein the shock absorber insert is located in the pocket.

5. The orthopedic walking boot of claim 1, wherein the shock absorber insert comprises a plurality of apertures, and wherein the outer sole extends through the apertures towards the insole plate.

6. The orthopedic walking boot of claim 1, wherein the shock absorber insert comprises a plurality of sections, and wherein each of the sections comprises a different stiffness.

7. The orthopedic walking boot of claim 1, wherein the shock absorber insert comprises a plurality of discrete shock absorber elements.

8. The orthopedic walking boot of claim 1, wherein the base comprises a plurality of apertures cooperating with the shock absorber insert.

9. The orthopedic walking boot of claim 1, further comprising:
one or more shock absorber pins between the base and the insole plate to provide shock absorption to the user's foot.

10. The orthopedic walking boot of claim 1, further comprising:
one or more shock absorber rings located on a surface of the insole plate to provide shock absorption to the user's foot.

11. An orthopedic walking boot, comprising:
a base to support a user's foot;
a support assembly extending along the lower leg from the base;
a rigid insole plate to support a bottom of the user's foot;
an outer sole; and
a shock absorber insert arranged between the insole plate and the outer sole to provide shock absorption to the user's foot.

12. The orthopedic walking boot of claim 11, wherein the shock absorber insert is arranged along a section of the base configured to support the user's plantar portion of the heel.

13. The orthopedic walking boot of claim 11, wherein the shock absorber insert comprises a plurality of ribs and the outer sole comprises a plurality of apertures, and wherein the ribs extend into the apertures.

14. The orthopedic walking boot of claim 11, wherein the shock absorber insert comprises a plurality of apertures, and wherein the outer sole extends through the apertures towards the insole plate.

15. The orthopedic walking boot of claim 11, wherein the shock absorber insert comprises a plurality of sections, and wherein each of the sections comprises a different stiffness.

16. The orthopedic walking boot of claim 11, wherein the shock absorber insert comprises a plurality of discrete shock absorber elements.

17. The orthopedic walking boot of claim 11, further comprising:
one or more shock absorber pins between the base and the insole plate to provide shock absorption to the user's foot.

18. The orthopedic walking boot of claim 11, further comprising:
one or more shock absorber rings located on a surface of the insole plate to provide shock absorption to the user's foot.
